(19) United States
(12) Patent Application Publication
(10) Pub. No.: US 2010/0051036 A1
(43) Pub. Date: Mar. 4, 2010

(54) Exercising Set for Pelvic Floor Muscles

(76) Inventors: Ira Kushnir, Lod (IL); Andrew Kolobov, Lod (IL)

Correspondence Address: The Law Office of Michael E. Kondoudis, 888 16th Street, N.W., Suite 800, Washington, DC, 20006, US

(21) Appl. No.: 12/202,834

(22) Filed: Sep. 2, 2008

(51) Int. Cl. A61F 6/06; A63B 23/00; A63B 21/065

(52) U.S. Cl. 128/834; 482/148; 482/105

(57) ABSTRACT

An exercising set and a method for enabling a user to exercise his/her pelvic floor muscles. The exercising set may include: at least one body member; an elongated member; at least one weight; and at least one fastening means. The elongated member may extend from the lower part of the body member. The body member may be inserted into the user's body cavity, where a lower part of the elongated member emerges outside the cavity. The fastening means may be connected to the weight, where the fastening means allows fastening of the weight to the emerging lower part of the elongated member, thereby enabling changing weights for the exercise without having to remove and re-insert the body member for each weight exercised. Methods of using the exercising set are disclosed.

### FIELD OF THE INVENTION

The present invention relates generally to the field of systems, methods and devices for physical exercising, and more particularly to systems, methods and devices for exercising of pelvic floor muscles.

### BACKGROUND OF THE INVENTION

An important function of the pelvic floor is to support organs that are located in the pelvis and to help supporting the breathing process. Dysfunctions in the pelvic floor muscles can cause various symptoms and related problems such as urinary and fecal incontinence, prolapses, detrusor instability and infections, inflammations and pain in the vaginal and rectum area.

Pregnancy may also cause various dysfunctions of the pelvic floor muscles leading to sexual disorders such as severe pain, anorgasmia and the like.

Pelvic floor dysfunction can often result from trauma, radiotherapy, incorrect toilet behavior, excessive coughing, heavy lifting, hormonal changes, neurological disorders, aging and may other causes.

Today various exercising methods such as Pilates, the Paula Method or Yoga enable many women and men to strengthen their pelvic floor muscles. Unfortunately, these exercises are hard to practice since it is extremely hard to know whether all the pelvic floor muscles are being exercised. To separate the inner pelvic muscles that are being practiced from other abdominal and hip muscles requires high expertise and there is no precision in the level of progress of the pelvic muscles build up.

Inner vaginal weights (often called “inner vaginal cones”) are devices that can be inserted into the patient's vagina in a similar way to inserting a tampon into the vagina of a women patient.

To practice the pelvic floor muscles the patient is required to simply hold the weight inside the vaginal cavity as long as she can. The weight of the cone is held by the patient's pelvic floor muscles, which hold the cone inside the vaginal cavity and thereby exercises these specific muscles.

The weight of the cones can be increased by replacing a lighter cone with a heavier one, for instance, to allow the patient to progress with the exercises by applying increasing loads upon the muscles.

This practice has proven extremely efficient in strengthening pelvic floor muscles, yet the device itself causes several problems.

One major problem with these cone weights is that due to their weight they can be stuck in a niche inside the patient's body cavity when the pelvic muscles are not exercised, in this situation the cones may stretch soft tissues inside that cavity, which in time, may lead to pelvic prolapse.

Each cone may be a single weight of a predefined weight, requiring the patient takes out a first cone of weight “W1” from her vagina and insert a new cone of weigh “W2” where W2>W1 to increase the force required by the pelvic floor muscles to hold the cone therein.

The cone may alternatively be comprised of a containing device enabling to open and close, where the weights can be removably placed therein, so that the patient can place a first weight “W1” in the device, insert the device into her vagina and then take it out, place another weight “W2” therein and insert the device again. These steps have to be repeated each time the patient needs to increase/decrease the weight in the cone.

Since the cone has to be inserted into the body cavity of the patient, the cone has to be repeatedly cleaned each time it is taken out and inserted into the patient's body cavity.

Since each exercising session can require exchanging more than one weight to increase and decrease the load applied on the pelvic floor muscles, this can be a tiresome job, which may lead the patient to either neglect the exercising altogether or skip the sterilizing process, which may be hazardous to the patient's health and hygiene.

### SUMMARY OF THE INVENTION

The present invention, according to some embodiments thereof, provides an exercising set for enabling a user such as a patient to exercise his/her pelvic floor muscles. The exercising set may comprise: at least one body member; an elongated member; at least one weight member; and at least one fastening means.

The body member may be inserted into the user's body cavity, which may be, for example, the user's vagina or rectum cavity, where the weights may fasten to the body member from outside the cavity of the user, enabling the user to replace and/or add and remove weights without having to re-insert the body member into the body cavity each time the weight held by the pelvic floor muscles have to be changed (either decreased or increased).

According to these embodiments of the invention, the elongated member, which may be, for example, a thread, may connect to and extends from a lower part of the body member. The body member may be inserted into the body cavity in a position where an upper part of the body member substantially faces upwards and the lower part and the elongated member extends therefrom face downwards towards the opening of the body cavity.

The length of the elongated member may be sufficient to allow a lower part of said elongated member to emerge from the body cavity outwardly, once the body member is fully inserted therein.

According to some embodiments of the invention, the fastening means may be connected to at least one edge of the at least one weight member enabling to fasten the weight member to the emerging lower part of the elongated member, once the body member is fully inserted in the user's body cavity.

The body member and elongated member may be a standard tampon with a thread serving as the elongated member, or alternatively a tampon-shaped element of sizes that may be equal or smaller than standard tampons to allow the user to apply pressure to hold the body member inside the body cavity preventing it from falling by exercising the pelvic floor muscles.

The exercising set may further comprise at least one disposable hygiene coating layer made of hygiene elastic isolating materials such as rubber or latex (condom materials) enabling the user to cover the body member before inserting it into the body cavity and remove the coating layer when the body member is removed from the body cavity. The coating layer may additionally be lubricated by lubricating materials suited for being inserted into the body cavity and touching the inner tissues in this cavity.

According to some embodiments of the invention, at least some of the weight member may comprise two fastening means a first fastening means connected at an upper edge of the weight member and a second fastening means connected at the lower edge of the weight member, where the upper first fastening means of one weight member enables fastening to the lower second fastening means of another weight member situated above it, enabling the user to add and remove weight members to and from the elongated member and thereby change the weight which is being held by the pelvic floor muscles during the exercise. Accordingly, a number of weight members may be added to one another may create a vertical row extending from the elongated member downwards.

According to some embodiments of the invention, the exercising set of may comprise a multiplicity of weight members of various weights.

According to some embodiments of the invention, the exercising set may further comprise an applicator enabling to facilitate the user in inserting the body member into the body cavity of the user by enabling the user to inset the body member into the applicator and guide the body member into the cavity thereby.

According to some embodiments of the invention, the surface of the body member may be smooth to facilitate in easily inserting of the body member into the user's cavity.

According to other embodiments of the invention, the exercising set may comprise: at least one body member; an elongated member; at least one weight holder; at least one weight member; and at least one fastening means, where the elongated member connects to and extends from the lower part of the body member. The body member may be adapted to be inserted into the body cavity of the user in a position where the upper part of the body member substantially faces upwards where the lower part and the elongated member extending therefrom faces downwards towards the opening of the body cavity, where the length of said elongated member is suitable for enabling an upper part of the elongated member to be inserted along with the body member inside the user's body cavity, while the rest of the elongated member, which is the lower part of the elongated member, emerges from the body cavity outwardly, once the body member is fully inserted therein. The weight holder may enable holding at least one weight member, where the weight-holder comprises fastening means connected thereto, enabling to fasten the weight holder to the emerging lower part of the elongated member.

Additionally, the weight holder may be a containing device enabling to contain at least one weight therein, where the weight holder comprises a first part and a second part, removably connected to one another via securing means. The first part may serve as a lid and the second part may serve as a cup, which is a containing device with an opening enabling the user to change the weight of the holder by changing the type and number of weight member(s) inserted therein.

According to some embodiments of the invention, the lower part of the weight holder, which serves a the cup may comprise at least one connecting member, connected to at the external lower edge of the cup, enabling to add and remove weight holders by connecting them to said connecting member.

### BRIEF DESCRIPTIONS OF THE DRAWINGS

The subject matter regarded as the invention will become more clearly understood in light of the ensuing description of embodiments herein, given by way of example and for purposes of illustrative discussion of the present invention only, with reference to the accompanying drawings, wherein:

FIG. 1 is a schematic illustration of an exercising set used by a user, for exercising the user's pelvic floor muscles, according to some embodiments of the invention;

FIG. 2 is a schematic illustration of an exercising set, for exercising a user's pelvic floor muscles, according to other embodiments of the invention;

FIG. 3 is a schematic illustration of an applicator with an exercising set, according to some embodiments of the invention; and

FIG. 4 is a schematic illustration of an exercising set with a weight-holder, according to other embodiments of the invention.

The drawings together with the description make apparent to those skilled in the art how the invention may be embodied in practice.

### DETAILED DESCRIPTIONS OF SOME EMBODIMENTS OF THE INVENTION

The present invention, in some embodiments thereof, provides a pelvic floor muscles exercising set 100 enabling a user 50 to exercise his/her pelvic floor muscles.

The user 50 mentioned in this document can be any female or male human being such as a patient who carries out the exercise, a trainer or any other person.

The exercising set 100 enables inserting a body member 110 into the user's 50 body cavity 55, where the body cavity 55 can be any inner cavity of the user 50 where the pelvic floors muscles can be exercised and where the body member 110 can be inserted to, either through the genitalia (female) or through the rectum (male or female). For example, the body cavity 55 can be the vaginal cavity, where the body member 110 is a tampon-shaped member enabling the user 50 to insert the body member 110 into the body cavity 55. The body member 110 may have little weight in comparison to the weight required for the exercise. The body member 110 may comprise an elongated member 115 such a thread (e.g. a cotton thread) which extends from the lower part of the body member 110.

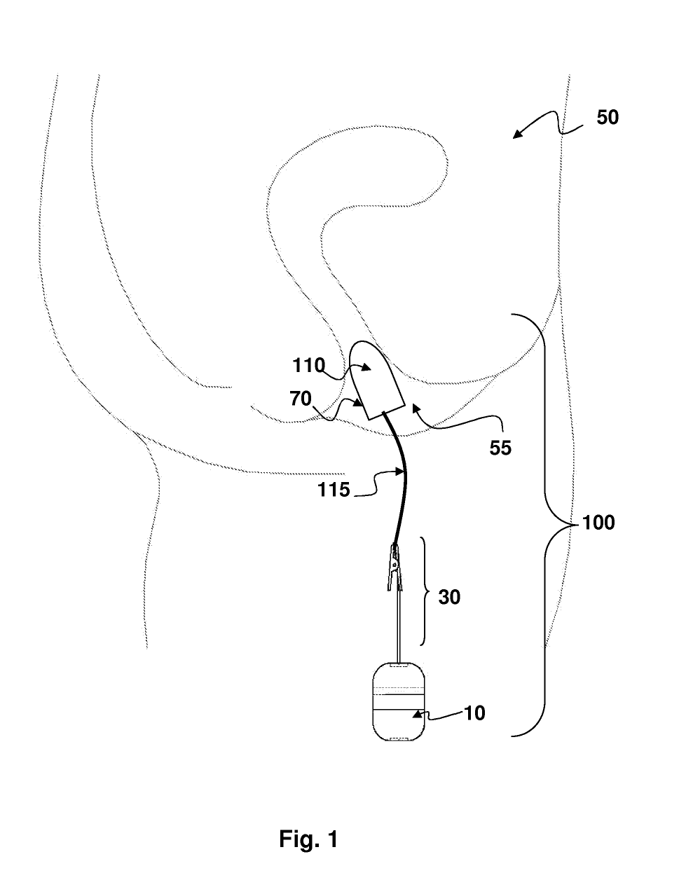

The elongated member's 115 length may be adapted to enable an upper part of the elongated member 115 to be inserted along with the body member 110 inside the user's body cavity 55, while allowing the rest of the elongated member 115 (meaning the lower part of the elongated member 115) to emerge from the body cavity 55, once the body member 110 is fully inserted therein, to allow fastening one or more weight members 10 (see FIG. 1) to the part of the elongated member 115 that is out of the cavity 55 and the user's 50 body. The body member 110 may be inserted into the body cavity 55 in a position where the upper part of the body member 110 substantially faces upwards and the lower part downwards towards the opening of the body cavity 55

The exercising set 100 may comprise one or more weight members 10 that can be connected to the elongated member 115 through various fastening means 30 from outside the body cavity 55 and body of the user 50.

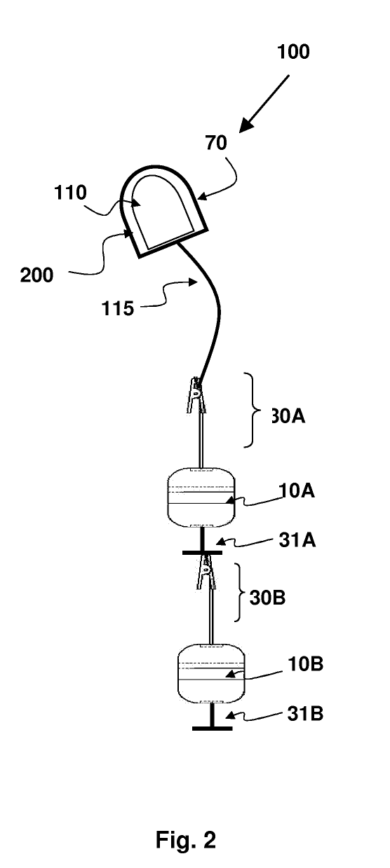

Once the body member 110 is securely seated inside the body cavity 55, the user 50 can increase or decrease the weight applied on the pelvic floor muscles by replacing the weight member 10 connected to the body member 110 elongated member 115 from outside the user's 50 body, or, alternatively, add or remove weight members 10A and 10B in a column connected to one another by connecting means. (See FIG. 2).

In this way, the body member 110 can be inserted only once in an exercising session into the user's 50 body cavity 55, while allowing the number of weight members 10A and 10B to be changed without removing the body member 110, which may only require sterilizing the body member 110 once per each exercising session, before it is inserted into the cavity 55.

The body members 110 may be especially designed for the exercising set 100 having different sizes which are smaller than the size of a tampon to allow the user to apply pressure using his/her pelvic floor muscles.

Alternatively or additionally, the set 100 may use a standard tampon(s) with a thread extending therefrom, where the weight members 10 are fastened to the thread of the tampon.

While the description below contains many specifications, these should not be construed as limitations on the scope of the invention, but rather as exemplifications of the preferred embodiments. Those skilled in the art will envision other possible variations that are within its scope. Accordingly, the scope of the invention should be determined not by the embodiment illustrated, but by the appended claims and their legal equivalents.

An embodiment is an example or implementation of the inventions. The various appearances of “one embodiment,” “an embodiment” or “some embodiments” do not necessarily all refer to the same embodiments. Although various features of the invention may be described in the context of a single embodiment, the features may also be provided separately or in any suitable combination. Conversely, although the invention may be described herein in the context of separate embodiments for clarity, the invention may also be implemented in a single embodiment.

Reference in the specification to “one embodiment”, “an embodiment”, “some embodiments” or “other embodiments” means that a particular feature, structure, or characteristic described in connection with the embodiments is included in at least one embodiments, but not necessarily all embodiments, of the inventions. It is understood that the phraseology and terminology employed herein is not to be construed as limiting and are for descriptive purpose only.

The principles and uses of the teachings of the present invention may be better understood with reference to the accompanying description, figures and examples. It is to be understood that the details set forth herein do not construe a limitation to an application of the invention. Furthermore, it is to be understood that the invention can be carried out or practiced in various ways and that the invention can be implemented in embodiments other than the ones outlined in the description below.

It is to be understood that the terms “including”, “comprising”, “consisting” and grammatical variants thereof do not preclude the addition of one or more components, features, steps, or integers or groups thereof and that the terms are to be construed as specifying components, features, steps or integers. The phrase “consisting essentially of”, and grammatical variants thereof, when used herein is not to be construed as excluding additional components, steps, features, integers or groups thereof but rather that the additional features, integers, steps, components or groups thereof do not materially alter the basic and novel characteristics of the claimed composition, device or method.

If the specification or claims refer to “an additional” element, that does not preclude there being more than one of the additional element. It is to be understood that where the claims or specification refer to “a” or “an” element, such reference is not be construed that there is only one of that element. It is to be understood that where the specification states that a component, feature, structure, or characteristic “may”, “might”, “can” or “could” be included, that particular component, feature, structure, or characteristic is not required to be included.

Methods of the present invention may be implemented by performing or completing manually, automatically, or a combination thereof, selected steps or tasks. The term “method” refers to manners, means, techniques and procedures for accomplishing a given task including, but not limited to, those manners, means, techniques and procedures either known to, or readily developed from known manners, means, techniques and procedures by practitioners of the art to which the invention belongs. The descriptions, examples, methods and materials presented in the claims and the specification are not to be construed as limiting but rather as illustrative only.

Meanings of technical and scientific terms used herein are to be commonly understood as by one of ordinary skill in the art to which the invention belongs, unless otherwise defined. The present invention can be implemented in the testing or practice with methods and materials equivalent or similar to those described herein.

Any publications, including patents, patent applications and articles, referenced or mentioned in this specification are herein incorporated in their entirety into the specification, to the same extent as if each individual publication was specifically and individually indicated to be incorporated herein. In addition, citation or identification of any reference in the description of some embodiments of the invention shall not be construed as an admission that such reference is available as prior art to the present invention.

FIG. 1 schematically illustrates an exercising set 100 for exercising the user's 50 pelvic floor muscles, comprising at least one body member 110 and at least one weight 10, according to some embodiments of the invention.

According to some embodiments of the invention, the body member 110 may have a rounded curved shape suited to fit in the body cavity 55 where it is designed to be inserted. For example, if the designated body cavity 55 is a woman's vagina lower pelvic area, than the shape of the body member 110 may be slightly narrower at the top part and slightly wider at the bottom part, in a manner that fits the inner side of a standard woman's vaginal genitalia or any user's rectal inner body cavity 55 (e.g. a pear shape or a tampon shape). Additionally, the tip of the bottom part of the body member 110 may be slightly coned or triangular to fit the opening of the woman's body cavity 55.

The body member 110 may have a smooth surface to allow easy and painless insertion of the body member 110 into the body cavity 55. Additionally, the set 100 may include lubrication material 70 (e.g. petroleum jelly) or any other lubrication for inner body tissue) for the user to put on the surface of the body member 110 to allow easy and smooth insertion of the body member 110 into the user's cavity 55.

According to some embodiments of the invention, as illustrated in FIGS. 1-4, the body member 110 may have an elongated member 115 such as a thread (e.g. a cotton thread similar to a tampon thread) extending from at one end to the lower part of the body member 110, where the fastening means 30 are designed to enable fastening to the thread.

According to embodiments illustrated in FIG. 1, the other end of the elongated member may be a free end, enabling to connect to it by holding this end.

According to the embodiments of the invention, illustrated in FIG. 2 each weight member 10A and/or 10B may enable connecting to fastening means 30A and/or 30B connected at the top edge of each weight 10A and/or 10B, enabling to fasten the weight members 10A and/or 10B to the elongated member 115 by fastening to its free end. For example, the fastening means 30 may comprise alligator or crocodile clips connected to the weight enabling to hold the free end of the elongated member 115 by tightly clutching to it.

The overall size of the body member 110 may be either in the range of standard tampon sizes or smaller. The smaller the body member 110 may be the harder it may be for the user 50 to hold it in the cavity 55, therefore the user 50 may be required to apply more muscles' strength to narrow pelvic floor muscles (e.g. annular muscles) in order to hold he body member 110 inside the body cavity 55, especially while the body member 110 is vertically fastened to one or more weights 10 having to also hold the weight of the weights pushing the body member 110 downwards and out of the body cavity 55.

FIG. 2 schematically illustrates an exercising set 100, comprising at least one body member 110 and a multiplicity of weight members 10A and 10B, according to other embodiments of the invention.

According to these embodiments the free end of the elongated member 115 may be fastened to the fastening means 30A at the upper end of a first weight member 10A. The first fastening means 30A may be, by way of non-limiting examples, hooks, crocodile fasteners or any other fasteners enabling to fasten to the free end of the elongated member 115.

Additionally, each weight member 10A and 10B may further connect to a second fastening means 31A and 31B connected at the bottom edge of each weight member 10A and 10B (e.g. hooks, where both the top and bottom fastening means 30 and 31 are hooks or compatible to be fastened to one another and to the connecting means 116 or the free end of the elongated member 115). The bottom and top fastening means 30 and 31 may allow chaining a vertical row of weight members 10A and 10B to one another to allow the user 50 to easily add and reduce weight to the exercise.

According to some embodiments of the invention, as illustrated in FIG. 2, the exercising set 100 may further comprise one or more disposable hygiene coating layers 200 enabling the user to cover the body member 110 before inserting it into the body cavity 55 and remove the coating layer 200 when the body member 110 is removed from the body cavity 55. The coating layer 200 may be made from elastic isolating materials, which may be sterilized, such as, by way of non-limiting examples, sterile latex or rubber (e.g. such as used for condoms). The coating layer 200 may be made from materials that enable isolating the body member 110 from the body cavity 55 preventing substances and particles from diffusing from one side of the layer 200 to another. Additionally, the coating layer 200 may be coated by lubrication materials 201 covering the outer surface of the coating layer 200 to allow easy insertion of the body member 110 and layer 200 into the body cavity 55.

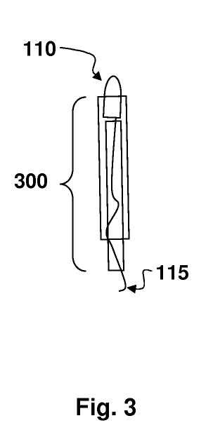

According to some embodiments of the invention, as illustrated in FIG. 3, an applicator 300 enabling to guide the body member 110 into the user's body cavity 55 may be added to the set 100. The applicator 300 may be a cylindrical device comprising a tube and a guide stick or cylinder enabling receiving the body member 110 at its top edge and guiding it into the body cavity 55 by placing the body member 110 at the edge of the applicator 300, inserting a portion of the applicator 300 into the body cavity 55 and pushing the guide stick or cylinder along the tube to allow pushing the body member 110 further inside the body cavity 55. The applicator 300 may be similar to a tampon applicator or any other medicine applicator suited for inserting medicine into the inner pelvic area as known in the art, where the applicator's 300 size is compatible to the size of the body member 110.

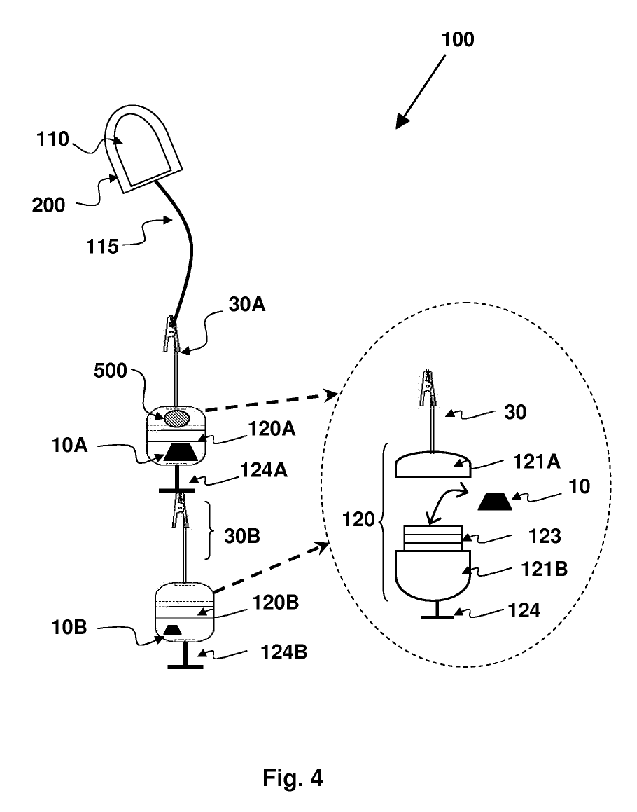

FIG. 4 schematically illustrates an exercising set 100, comprising: at least one body member 110; a first and a second weight-holders 120A and 120B respectively and a multiplicity of weight members 10A and 10B, according to some embodiments of the invention.

According to some embodiments of the invention, as illustrated in FIG. 4, the body member 110 may comprise an elongated member 115 such as a thread (e.g. a cotton thread similar to a tampon thread), which is connected at one end to the lower part of the body member 110 and to the first weight-holder 120A at the other end.

According to these embodiments, as illustrated in FIG. 4, each weight-holder 120A and 120B may enable holding at least one weight member 10A and/or 10B respectively, where the first weight-holder 120A may comprise a first upper fastening means 30A such as crocodile clips (see FIG. 4) to fasten to the elongated member 115 of the body member 110.

The weight-holder 120 may be either a containing device enabling to hold one or more weight members 10A therein, or a holder of any kind known in the art such as a cup-shaped element with a fastening means 30 integrally connected to the upper edge of the element etc. as known in the art.

According to some embodiments of the invention, as illustrated in FIG. 4, each of the weight holders 120A and/or 120B may be a containing device comprised of a first “lid” part 121A and a second “cup” part 121B, removably connected to one another via securing means 123 (e.g. by a male-female screwing mechanism enabling to screw the lid 121A to the cup 121B and thereby secure the weight 10 inside the weight holder 120 as shown in FIG. 4), where the first part is a lid and the second part is a cup. The user 50 may be able to add and remove weights 10 to the weight-holder 120 by opening the lid placing and/or removing the weight 10 and securing the lid back to the cup.

Each weight-holder 120A and/or 120B may further connect to a connecting member 124A and 124B at the bottom edge of each holder 120A and 120B to allow attaching, for example, the second weight-holders 120B to the first weight holder 120A in a vertical row, by fastening the upper fastening means 30A of the lower second weight holder 120B to the lower connecting member 124A of the upper first weight holder 120A.

According to some embodiments of the invention, as illustrated in FIG. 3, the exercising set 100 may further comprise a vibrating device 500 assembled to the set 100 in any way that allows the device 500 to vibrate the weight members 10A and 10B and/or weight holder 120A and/or 120B being attached to an element of the set 100 that is designed to be situated externally to the body cavity 55 of the user 50. For example, the vibrating device 500 may be situated inside the first upper weight holder 120A or attached to it.

While the invention has been described with respect to a limited number of embodiments, these should not be construed as limitations on the scope of the invention, but rather as exemplifications of some of the preferred embodiments. Those skilled in the art will envision other possible variations, modifications, and applications that are also within the scope of the invention. Accordingly, the scope of the invention should not be limited by what has thus far been described, but by the appended claims and their legal equivalents.

What is claimed is:

1. An exercising set for enabling a user to exercise his/her pelvic floor muscles, said exercising set comprising: at least one body member; an elongated member; at least one weight member; and at least one fastening means,
wherein said elongated member is adapted to be connected to and extends from the lower part of the body member,
wherein the body member is adapted in use to be inserted into the body cavity of the user, in a position where the upper part of the body member substantially faces upwards and the lower part and the elongated member extending therefrom faces downwards towards the opening of the body cavity,
wherein the length of said elongated member is sufficient to allow a lower part of said elongated member to emerge from the body cavity outwardly, once the body member is fully inserted therein, and
wherein said fastening means is adapted in use to be connected to at least one edge of the weight member enabling to fasten the weight to the emerging lower part of the elongated member, once the body member is fully inserted in the user's body cavity.

2. The exercising set of claim 1, further comprising at least one disposable hygiene coating layer enabling the user to cover the body member before inserting it into the body cavity and remove the coating layer when the body member is removed from the body cavity, wherein said coating layer is made from elastic isolating materials.

3. The exercising set of claim 1, wherein at least some of the weight members connect to a first and a second fastening means, wherein the first fastening means is connected to an upper edge of the weight member and the second connects to the lower edge of the weight, wherein the upper first fastening means of one weight enables fastening to the lower second fastening means of another weight situated above it, enabling the user to add and remove weights to and from the elongated member, and
wherein a number of weights added to one another create a vertical column extending from the elongated member.

4. The exercising set of claim 1, wherein said set comprises a multiplicity of weight members of various weights.

5. The exercising set of claim 1, further comprising at least one weight holder enabling holding at least one weight member, wherein said weight-holder connects to at least one fastening means enabling to fasten to the emerging lower part of the elongated member.

6. The exercising set of claim 5, wherein said weight holder is a containing device enabling to contain at least one weight member therein, wherein said weight holder comprises a first part and a second part, removably connected to one another via securing means, and
wherein the first part is a lid and the second part is a cup, enabling the user to change the weight of the holder by changing the weight members inserted therein.

7. The exercising set of claim 6, wherein the lower part of the weight holder, which serves as the cup, comprises at least one connecting member, connected to at the external lower edge of the cup, enabling to add and remove other weight holders and weights by connecting them to said connecting member.

8. The exercising set of claim 1, further comprising an applicator, enabling to facilitate the user in inserting the body member into the body cavity by enabling the user to inset the body member into the applicator and guide the body member into the cavity.

9. The exercising set of claim 1, wherein said elongated member is a thread.

10. The exercising set of claim 9, wherein said thread is a made from cotton.

11. The exercising set of claim 1, wherein the surface of the body member is smooth to facilitate in easily inserting of the body member into the user's cavity.

12. The exercising set of claim 1, wherein said body member is a tampon and said elongated member is a tampon thread extending from a lower edge of the tampon.

13. An exercising set for enabling a user to exercise his/her pelvic floor muscles, said exercising set comprising: at least one body member; an elongated member; at least one weight holder; at least one weight member; and at least one fastening means,
wherein said elongated member is adapted to connect to and extends from the lower part of the body member,
wherein the body member is adapted to be inserted into the body cavity in a position where the upper part of the body member substantially faces upwards and the lower part and the elongated member extends therefrom face downwards towards the opening of the body cavity,
wherein the length of said elongated member is suitable for enabling an upper part of the elongated member to be inserted along with the body member inside the user's body cavity, while the rest of the elongated member, which is the lower part of the elongated member, emerges from the body cavity outwardly, once the body member is fully inserted therein, and
wherein said at least one weight holder enables holding at least one weight, wherein said weight-holder comprises fastening means enabling to fasten to the emerging lower part of the elongated member.

14. The exercising set of claim 13, wherein said weight holder is a containing device enabling to contain at least one weight member therein, wherein said weight holder comprises a first part and a second part, removably connected to one another via securing means, and
wherein the first part is a lid and the second part is a cup, enabling the user to change the weight of the holder by changing the weight members inserted therein.

15. The exercising set of claim 14, wherein the lower part of the weight holder, which serves a the cup, comprises at least one connecting member, connected to at the external lower edge of the cup, enabling to add and remove weight holders and weight members by connecting said weight holders through said connecting members and fastening means.

16. The exercising set of claim 13, further comprising a vibrating device enabling to vibrate, which connects to the set in a way that allows the device to vibrate the weight members, wherein said vibrating device is attached to an element of the set that is designed to be situated externally to the body cavity of the user.

17. The exercising set of claim 16, wherein said vibrating device is inside the weight holder, enabling to vibrate the weight holder and thereby the weights therein.

18. A set comprising at least one weight member comprising at least one fastening means connected thereto, wherein said fastening means is designed to be fastened to an emerging part of an elongated member that extends from a body member that is adapted to be inserted into a body cavity of a user.

19. The set of claim 18, wherein said body member is a tampon having a thread as the extending elongated member, where a part of the thread emerges from the user's body when said tampon body is inserted into the user's body cavity, enabling to fasten the weight member's fastening means to said emerging part of the thread.

20. The set of claim 18, wherein each weight member in said set has a different weight to allow the user to change the weight applied on said elongated member.
